(19) United States
(12) Patent Application Publication
(10) Pub. No.: US 2024/0138769 A1
(43) Pub. Date: May 2, 2024

(54) IMPLANTABLE SENSING DEVICE CONFIGURED FOR MOUNTING ON OUTER SURFACE OF BONE

(71) Applicant: Zimmer, Inc., Warsaw, IN (US)

(72) Inventors: Kevin Miller, Wakarusa, IN (US); Michael Francis Kovacs, Winona Lake, IN (US); Raymond C. Parisi, Wakarusa, IN (US); Andrew Scott Payton, Warsaw, IN (US); Trevor Paul Brickner, Goshen, IN (US)

(21) Appl. No.: 18/494,526

(22) Filed: Oct. 25, 2023

Related U.S. Application Data
(60) Provisional application No. 63/419,926, filed on Oct. 27, 2022.

(51) Int. Cl. A61B 5/00; A61B 5/07

(52) U.S. Cl. CPC A61B 5/6878; A61B 5/076; A61B 5/4576; A61B 5/4851; A61B 5/6883

(57) ABSTRACT

A sensing device may include a housing and one or more features configured to couple the housing against the outer surface of the bone of the patient. A sensing device may include onboard electronics carried by the housing, the onboard electronics comprising: communication circuitry, one or more sensors configured to generate data regarding one or more characteristics of the patient; and processing circuitry configured to activate the communication circuitry to send the generated data to a device outside the patient.

### CLAIM OF PRIORITY

This application claims the benefit of U.S. Provisional Patent Application Ser. No. 63/419,926, filed on Oct. 27, 2022, the benefit of priority of which is claimed hereby, and which is incorporated by reference herein in its entirety.

### BACKGROUND

The shoulder joint is a complex joint with the scapula, clavicle, and humerus all coming together to enable a wide range of movement, at least in a properly functioning joint. In a properly functioning shoulder joint, the head of the humerus fits into a shallow socket in the scapula, typically referred to as the glenoid. Articulation of the shoulder joint involves movement of the humeral head in the glenoid, with the structure of the mating surfaces and surrounding tissues providing a wide range of motion.

The shoulder joint can undergo degenerative changes caused by various issues, such as rheumatoid arthritis, osteoarthritis, rotator cuff arthroplasty, vascular necrosis, or bone fracture. When severe joint damage occurs, and no other means of treatment is found to be effective, a total, partial, or reverse shoulder replacement or reconstruction may be necessary. Total shoulder replacements can involve a humeral prosthetic, including a stem and a head portion used to replace the natural humeral head. Total shoulder replacements will also typically include resurfacing of the glenoid with a prosthetic implant. The glenoid implant will generally include an articulating cup shaped to receive the prosthetic humeral head. A reverse shoulder replacement (arthroplasty) involves a different set of humeral and glenoid replacement prosthetics. In a reverse shoulder replacement, the humeral component includes a cup-shaped articular surface attached to a stem implanted into the humerus, while a spherical glenoid component is used to provide an articular surface for the humeral cup.

Postoperative care may include immobility of a joint ranging from weeks to months, physical therapy, or occupational therapy. Physical therapy or occupational therapy may be used to help the patient with recovering strength, everyday functioning, and healing. Current techniques involving immobility, physical therapy, or occupational therapy may not monitor or adequately assess range of motion or other patent characteristics after surgical intervention.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic illustration of a shoulder joint having a sensing device installed within a human body on an outer surface of a humerus adjacent the shoulder joint according to an example of the present application.

FIG. 2 is a schematic illustration of the shoulder joint having an orthopedic implant in addition to a sensing device with the sensing device implanted on the outer surface of the humerus according to an example of the present application.

FIG. 3A is an anterior-posterior plan view illustrating potential mounting areas for the sensing device on the outer surface of the humerus according to an example of the present application.

FIG. 3B is a medial-lateral plan view of a lateral side of the humerus illustrating potential mounting areas for the sensing device on the outer surface of the humerus according to an example of the present application.

FIG. 3C is a medial-lateral plan view of a medial side of the humerus illustrating potential mounting areas for the sensing device on the outer surface of the humerus according to an example of the present application.

FIG. 4A shows an example of a sensing device implanted on a biceps groove of the humerus according to an example of the present application.

FIG. 4B is a highly schematic view of various components of the sensing device of FIG. 4A according to an example of the present application.

FIGS. 4C and 4D show the sensing device of the FIGS. 4A and 4B from different perspectives in isolate from the humerus according to examples of the present application.

FIGS. 5A and 5B show another example of a sensing device configured for implantation on the outer surface of the humerus according to an example of the present application.

FIGS. 6A and 6B show the sensing device of FIGS. 5A and 5B implanted at a lateral side area on the outer surface of the humerus according to an example of the present application.

FIGS. 7A and 7B show another example of a sensing device configured for implantation on the outer surface of the humerus according to an example of the present application.

FIGS. 8A and 8B show the sensing device of FIGS. 7A and 7B implanted at medial to lesser tuberosity on the outer surface of the humerus according to an example of the present application.

FIGS. 9A and 9B show another example of a sensing device configured for implantation on the outer surface of the humerus according to an example of the present application.

FIGS. 10A and 10B show the sensing device of FIGS. 9A and 9B implanted at a medial side area on the outer surface of the humerus according to an example of the present application.

FIGS. 11A-11C show another example of a sensing device configured for implantation on the outer surface of the scapula such as the coracoid according to an example of the present application.

FIGS. 12A and 12B show the sensing device of FIGS. 11A-11C implanted at base of the coracoid on the outer surface of the scapula according to an example of the present application.

FIGS. 13A-13C show another example of a sensing device configured for implantation on the outer surface of the scapula such as the coracoid according to an example of the present application.

FIGS. 14A and 14B show the sensing device of FIGS. 13A-13C implanted at a proximal side of the coracoid on the outer surface of the scapula according to an example of the present application.

FIG. 15 depicts a system of sensors including at least two sensors with one on the humerus and one on the scapula, the system measure abduction of the shoulder joint according to an example of the present application.

FIG. 16A shows a clinical application using the system of FIG. 15 where the shoulder joint is health (no tears in a rotator cuff) and abduction is measured using the system of FIG. 15 according to an example of the present application.

FIG. 16B shows a clinical application using the system of FIG. 15 where the shoulder joint has tears to the rotator cuff and abduction is measured using the system of FIG. 15 according to an example of the present application.

### DETAILED DESCRIPTION

This disclosure relates generally to a sensing device configured to be implanted into a human body. In particular, the sensing device can be configured to be implanted on an outer surface (external surface) of the bone of the patient, for example. However, the term “outer surface of the bone” as used herein contemplates mounting of the sensing device to other tissue on the exterior of the bone such as cartilage, muscle, ligament, etc. Thus, the term “outer surface of the bone” as used herein should not be limited to only bone tissue. The sensing device can be fixated to the bone or other tissue including soft tissue. In most of the examples discussed herein, the sensing device is implanted to be subcutaneous. However, certain portions of the sensing device such a lead, if utilized, can be transdermal. The term “outer surface of the bone” contemplates any mounting location along the extent of the bone including the epiphysis, the metaphysis and/or the diaphysis of a long bone. While it is contemplated that the sensing device can be configured for insertion within bone in some examples, the examples discussed and illustrated herein contemplate mounting to the outer surface of the bone as previously defined. The term “bone” as used herein is not limited to the humerus but can include any applicable bone of the body including the tibia, femur, scapula, etc.

The sensing devices disclosed herein are separate components from an orthopedic implant that replicates a portion of the patient's joint. Thus, the sensing device is spaced from such implants and is not configured for insertion into and/or is not part of the orthopedic implants. However, it is contemplated in some examples that one or more sensors of the sensing device can be placed on or in the orthopedic implant for sensing one or more characteristics of the patient. It is contemplated that in instances of use of the one or more sensors with the orthopedic implant, the other components of the sensing device (e.g., a housing, communication circuitry, battery, processing circuitry, memory, etc.) would be implanted on the outer surface of the bone spaced from the orthopedic implant. Thus, the sensing device including the components discussed above would not be housed in a part of the orthopedic implant such as the stem as previously known in the art and discussed in, for example, U.S. Provisional Patent Application No. 63/319,552, entitled “SHOULDER STEM WITH MODULAR SENSOR”, filed Mar. 14, 2022, the entire contents of which are incorporated by reference in their entirety. While the sensing location and technology discussed in the Provisional Patent Application No. 63/319,552 is adequate, such sensing location has challenges with regard to recharging batteries and the transmission of data, for example. The present inventors also recognize that modifying implant designs to have onboard electronics including one or more sensors with controllers and other electronics can be expensive and can negatively impact the strength of the implant design as material must be removed to create a cavity for a sensor(s) and/or other electronics. Retrofitting orthopedic implants to add sensors or replace a discharged battery generally involves disturbing the joint. The present inventors have recognized that a sensing device implanted on the outer surface of the bone of the patient can be more accessible for replacement, recharging of the battery and can be more effective for the transmission of data. Obtaining data via transmission from a sensing device at a location on the outside of the bone has less challenges from potential interference and can allow components such as the antenna to be reduced in size. Further benefits include cost savings from not having to perform additional surgery and/or modify existing implants to make them smart. The present inventors further recognize that the sensing device can be utilized independent of any use of an orthopedic implant. Thus, the sensing device could be implanted on the patient for joint health or other health monitoring purposes prior to an arthroplasty or other surgical procedure. Thus, the sensing device can be used for preoperative as well as postoperative purposes.

Although the examples are described herein in reference to mounting on the outer surface of the bone (e.g., the humerus) and reference an orthopedic implant for a shoulder arthroplasty, the apparatuses, systems, techniques and methods discussed herein are not so limited and can be used in other anatomic locations such as adjacent other joints such as the spine, knee, hip, ankle, wrist or the like.

The term “proximal” refers to the general orientation of the side and/or surface when the sensing device is implanted in the bone. Thus, “proximal” refers to a direction or location generally in the direction of or toward the head of a patient, and “distal” refers to the opposite direction of proximal, i.e., away from the head of a patient. As used herein, the terms “anterior” and “posterior” should be given their generally understood anatomical interpretation. Thus, “posterior” refers to a location or direction generally toward a rear of the patient. Similarly, “anterior” refers to a location or direction generally toward a front of the patient. Thus, “posterior” refers to the opposite direction of “anterior.” Similarly, the terms “medial” and “lateral” should be given their generally understood anatomical interpretation. “Medial” refers to the more inward facing (inner part) of the prosthesis (when in the implanted orientation) and “lateral” refers to the outer part or outward facing part. “Medial” refers to the opposite direction of “lateral”.

The above discussion is intended to provide an overview of subject matter of the present patent application. It is not intended to provide an exclusive or exhaustive explanation of the invention. The description below is included to provide further information about the present patent application.

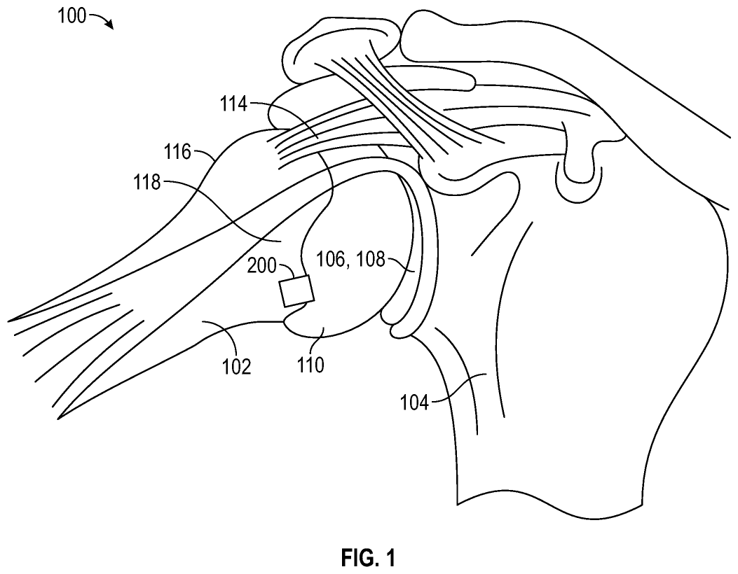

FIG. 1 illustrates a shoulder joint 100 with several ligaments stripped away. As shown, the shoulder joint 100 includes a humerus 102 and a scapula 104 that has a glenoid 106 with a socket 108 for interacting with a humeral head 110 of humerus 102. Humeral head 110 can articulate within socket 108 to allow for normal motion of the shoulder joint 100. A critical group of muscles and tendons for maintaining humeral head 110 within socket 108 is the rotator cuff 114. When rotator cuff 114 becomes damaged (e.g., torn or degraded), normal shoulder joint function can become compromised. Since rotator cuff 114 is not functioning correctly, humeral head 110 can become dislodged from glenoid 106 during normal motion. Alternatively, disease can degenerate the bone or soft tissue of the humeral head 110 and/or scapula 104. These can cause pain and/or can negatively impact shoulder joint function.

FIG. 1 shows a sensing device 200 implanted on an outer surface 116 of the humerus 102 such as at a metaphysis region 118 of the humerus 102. Examples of the sensing device 200 are described subsequently. The sensing device 200 can be used for preoperative sensing of one or more characteristics of the patient such as those indicative of a health of the rotator cuff 114, and/or can monitor the joint for other characteristics such as: range of motion of the shoulder joint, infection, joint tension, force, pH, temperature, and/or other criteria.

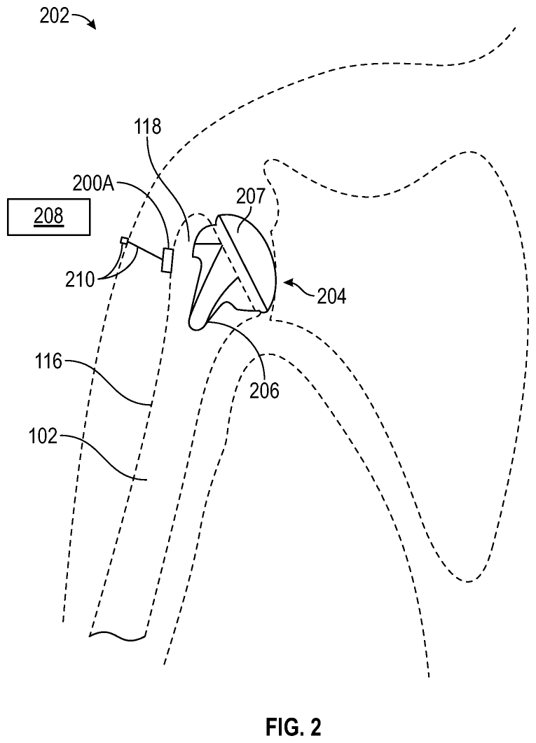

FIG. 2 illustrates a schematic diagram of a system 202 including an orthopedic implant 204 and a sensing device 200A. Both the orthopedic implant 204 and the sensing device 200A comprise separate implantable devices as discussed above. Thus, the sensing device 200A is spaced from the orthopedic implant 204. The orthopedic implant 204 can include a humeral implant component 206 and a head component 207. The sensing device 200A can be similar or differ in construction from the sensing device 200 of FIG. 1. The sensing device 200A can be configured for postoperative sensing of one or more characteristics of the patient such as those indicative of joint movement and/or pain such as, but not limited to, a range of motion of the shoulder joint, force, pH, temperature, implant loosening, fracture, infection, joint tension and/or other criteria.

The orthopedic implant 204 can be a standard (non-smart) implant designed for a total shoulder replacement procedure, revision shoulder replacement procedure or a partial shoulder replacement procedure, for example. The example of FIG. 2 shows a stem-less or stem-free design for the orthopedic implant 204. The joint replacement procedures contemplated include an anatomic shoulder arthroplasty or reverse shoulder arthroplasty (RSA) procedure.

As shown in FIG. 2, a proximal side of the humeral implant component 206 can interface with the head component 207. The head component 207 can couple with the humeral implant component 206 using known mechanisms such as mating taper features or the like. The head component 207 can be semi-spherical or otherwise shaped to replicate the head of the humerus (which is resected during the joint replacement procedure). Thus, the orthopedic implant 204, in particular, the head component 207, is configured to replicate a portion of the humerus 102 that forms the shoulder joint 100 of the patient. The orthopedic implant 204, in particular the humeral implant component 206, is configured for coupling to the resected surface of the humerus 102 as shown.

The humeral implant component 206 can be configured in the manner of the Sidus® Stem-Free Shoulder prosthesis or Comprehensive® Nano Stemless Shoulder commercially available and manufactured by Zimmer Biomet Inc., of Warsaw Indiana. The orthopedic implant 204 designs shown in the FIGURES are purely exemplary and are provided merely to facilitate practitioner understanding. The orthopedic implant 204 can be a standard implant, and thus, does not have sensors or other electronic equipment built into the device. This can make the orthopedic implant 204 less expensive as compared to smart implants that have sensors and/or other electronics.

Examples of the sensing device 200A are shown and discussed in further detail subsequently and further examples not specifically shown are contemplated. In brief, the sensing device 200A can include an antenna (or wired communication and/or charging element such as a lead 210) and other onboard electronic components including one or more sensors. As shown in FIG. 2, the sensing device 200A is a separate device from the orthopedic implant 204 and is spaced therefrom. The sensing device 200A is implanted on the outer surface 116 of the humerus 102 such as at the metaphysis region 118 of the humerus 102. In addition to the orthopedic implant 204 and the sensing device 200A, the system 202 can include an electronic device external of the patient such as a controller 208.

The controller 208 can include one or more processors, microprocessors, microcontrollers, electronic control modules (ECMs), electronic control units (ECUs), programmable logic controller (PLC), or any other suitable means for electronically communicating with the sensing device 200A. The controller 208 can be configured to operate according to a predetermined algorithm or set of instructions for communicating with sensing device 200A. Such an algorithm or set of instructions can be stored in a database, can be read into an on-board memory of the controller 208, or preprogrammed onto a storage medium or memory accessible by the controller 208, for example, in the form of a floppy disk, hard drive, optical medium, random access memory (RAM), read-only memory (ROM), or any other suitable computer-readable storage medium commonly used in the art (each referred to as a “database”), which can be in the form of a physical, non-transitory storage medium.

The controller 208 can be in electrical communication or connected to a display (not shown), or the like, and various other components, or multiple smart implants, like sensing device 200A. By way of such connection, the controller 208 can receive data pertaining to the rehabilitation or diagnostic data stored within the sensing device 200A. In response to such input, the controller 208 can perform various determinations and transmit output signals corresponding to the results of such determinations or corresponding to actions that need to be performed, such as alerting the doctor of any recommended physical therapy exercises or alerting the doctor to any potential patient characteristics indicative of joint health (e.g., implant loosening, fracture, infection, joint tension, swelling, temperature at or adjacent the joint, pH at or adjacent the joint, range of motion data, etc.).

The controller 208, including a human-machine interface, can include various output devices, such as screens, video displays, monitors and the like that can be used to display information, warnings, data, such as text, numbers, graphics, icons, and the like, regarding the status of the sensing device 200A. The controller 208, including the human-machine interface, can additionally include a plurality of input interfaces for receiving information and command signals from various sensors associated with the sensing device 200A and a plurality of output interfaces for sending control signals to sensing device 200A. Suitably programmed, the controller 208 can serve many additional similar or wholly disparate tasks/purposes.

The example of FIG. 2 shows the sensing device 200A having the lead 210. The lead 210 is a transdermal lead passing from the sensing device 200A (mounted on the outer surface 116 of the humerus 102 at a subcutaneous location). The lead 210 can pass through the soft tissue including the skin and can extend to external of the epidermis, for example. The lead 210 can be configured for electronic communication with the controller 208, for example. This can be via wired connection. Additionally, or alternatively, the lead 210 can be configured for recharging of a battery of the sensing device 200A.

Although FIG. 2 illustrates an example using a shoulder implant and system 202 for monitoring the shoulder joint, it is contemplated that sensing device 200A can be utilized with or without an arthroplasty implant or other implant. Furthermore, the sensing device 200A can be used with any arthroplasty implant (stemmed/stemless) including those utilized for repair of fractures, those used for revision or partial arthroplasty procedures, those configured for limb salvage or indeed in other procedures. The sensing device 200A could utilized on its own after procedures such as sports repair of torn or damaged soft tissue to monitor joint health, healing, pain and/or other criteria.

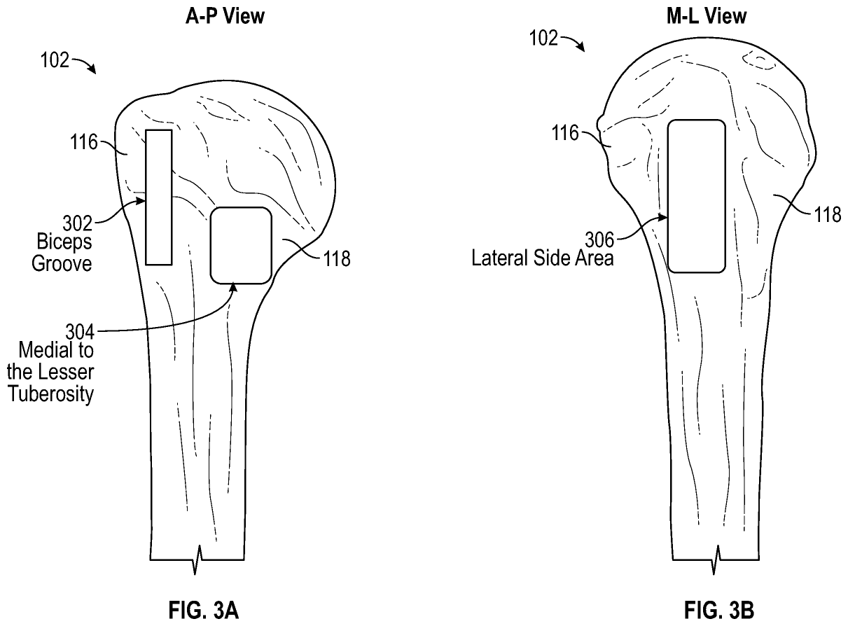

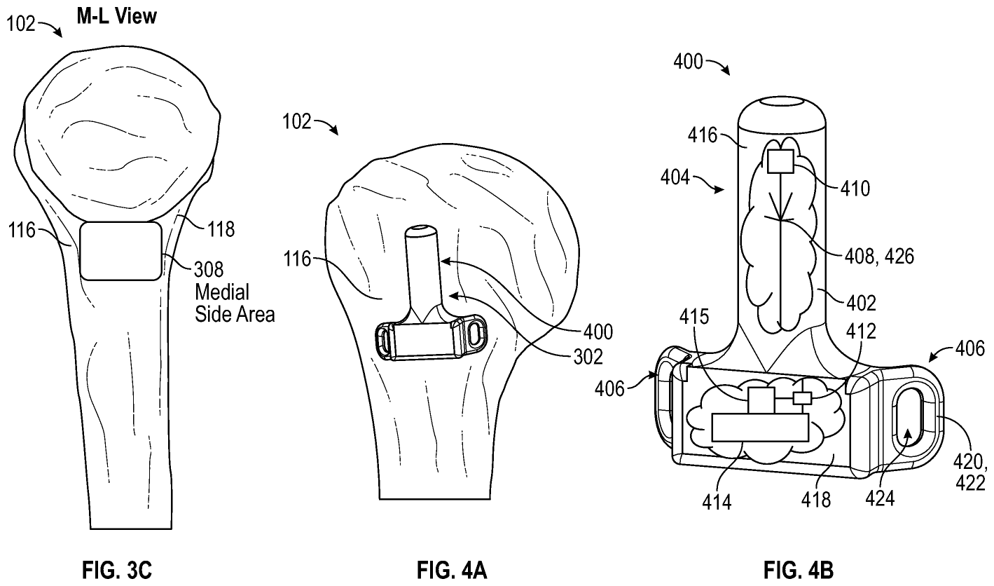

FIGS. 3A-3C illustrate a proximal portion of the humerus 102 from various views. FIG. 3A shows an anterior-posterior view of an anterior side of the humerus 102. The outer surface 116 of the humerus 102 is illustrated. Potential mounting locations for the sensing device 200 and 200A (FIGS. 1 and 2) such as at the metaphysis region 118 are indicated. However, other mounting locations such as on the diaphysis or other areas of the metaphysis region 118 are also contemplated. The potential mounting locations shown in FIG. 3A include a biceps groove 302 and a medial to lesser tuberosity 304.

A biceps release is typically performed during a total shoulder arthroplasty and a revision shoulder arthroplasty. A repair of the biceps is typically performed via tenodesis below the biceps groove 302. Thus, the biceps groove 302 would be clear to receive the sensing device 200 and/or 200A (FIGS. 1 and/or 2). The medial to lesser tuberosity 304 is an area easily accessible in the deltopectoral technique. Thus, the medial to lesser tuberosity 304 is another area on the outer surface 116 for potential mounting of the sensing device.

FIG. 3B shows a medial-lateral view of a lateral side of the humerus 102. This view shows a lateral side area 306 that would be available for mounting of the sensing device 200 and/or 200A (FIGS. 1 and/or 2). The lateral side area 306 can extend from adjacent a humeral head to a humeral neck. This lateral side area 306 can be distal of the supraspinatus insertion point and tucked under the deltoid, for example.

FIG. 3C shows a medial-lateral view of a medial side of the humerus 102. This view shows a medial side area 308 can be below the articular margin. The medial side area 308 could use an open window in the rotator cuff where interference is less likely.

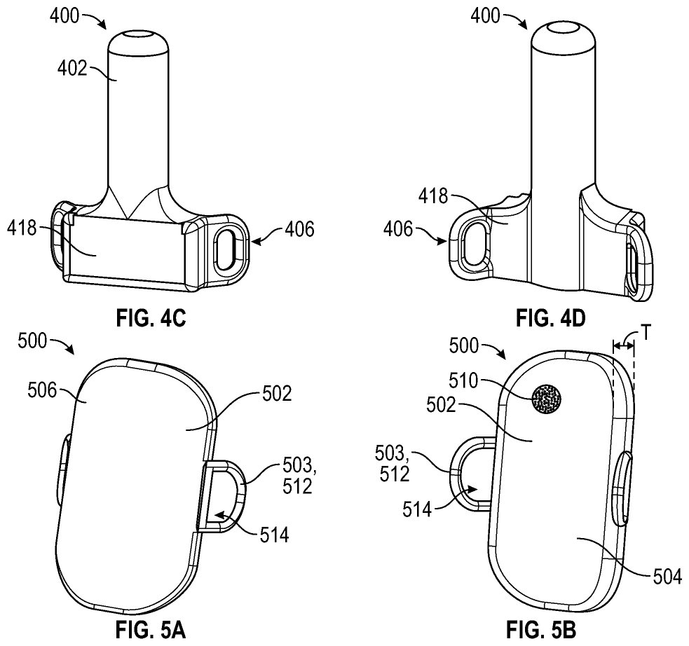

FIG. 4A shows a schematic view of an example of a sensing device 400 implanted on the outer surface 116 of the humerus 102 including at the biceps groove 302. FIGS. 4B-4D show additional views of the sensing device 400 in isolation from the humerus. As shown in FIG. 4A, the sensing device 400 can be configured for mounting to the outer surface 116 of the humerus 102 including at/within the biceps groove 302. Thus, it can be sized and shaped accordingly as further discussed herein.

Turning to FIG. 4B, the sensing device 400 can include a housing 402. FIG. 4B shows portions of the housing 402 broken away to show internal components such as onboard electronics 404 carried by the housing 402 of the sensing device 400. The sensing device 400 can further include one or more mounting features 406. The onboard electronics 404 can include communication circuitry 408, one or more sensors 410, processing circuitry 412, a battery 414 and a memory 415.

The housing 402 can include a cylindrical portion 416 and a main body portion 418. The cylindrical portion 416 can extend outward from the main body portion 418 such as in a generally proximal direction (or direction dictated by the orientation of the biceps groove 302) when mounted to the outer surface 116 of the bone as shown in FIG. 4A. The cylindrical portion 416 can be configured to rest in the biceps groove 302 as shown in FIG. 4A. The cylindrical portion 416 can be configured to carry some of the onboard electronics 404 (e.g., the communication circuitry 408 (including an antenna) and the one or more sensors 410). The main body portion 418 can be generally rectangularly box shaped feature having a longer medial-lateral extent than a proximal-distal extent as shown in FIG. 4A when implanted on the humerus 102. The main body portion 418 can be relatively larger in size than the cylindrical portion 416. The main body portion 418 can be configured to carry the processing circuitry 412, the battery 414 and the memory 415, for example. The main body portion 418 can be arranged to extend generally medial-lateral with a major dimension on the outer surface 116 of the humerus 102 as shown in FIG. 4A. Thus, the cylindrical portion 416 can have a longitudinal dimension that is generally transverse to a longitudinal dimension of the main body portion 418. However, other arrangements for the cylindrical portion 416 and the main body portion 418 are contemplated. The term “generally” as used above contemplates other orientations for implantation as desired.

The bone-interfacing side of the sensing device 400 can be shaped based on average anatomy derived from two-dimensional or three-dimensional scans of humeri using X-ray, MRI, CT, ultrasound or other imaging techniques. As an example, the average size of the biceps groove could be used to determine the geometry of the sensing device that registers this anatomy. It is contemplated that a diameter of the cylindrical portion 416 can be between 3 mm and 8 mm but such size can vary depending upon the average size of the biceps groove or the actual size of the biceps groove for the individual patient. Thus, the diameter of the cylindrical portion 416 can be based on data collected on the size of the biceps groove from a patient population or from the specific patient receiving the device.

The main body portion 418 can be integral with or can comprise a separate piece from the cylindrical portion 416. The one or more mounting features 406 can be positioned medial and/or lateral of the main body portion 418 and the cylindrical portion 416, for example. The one or more mounting features 406 can be configured to couple the housing 402 against the outer surface 116 of the humerus 102 as shown in FIG. 4A. In particular, the one or more mounting features 406 can be configured as any one or combination of fins, threads, tangs, prongs, tabs, hooks, loops, arms, apertures (e.g., slots, holes, etc.) or other known mechanical coupling features and can be configured for mechanical coupling to the external surface of the bone via a fastener, suture, cable, anchor or other component as discussed further below. As shown in FIG. 4A, the one or more mounting features 406 are each shaped as an arm 420 forming a loop 422 with an aperture 424. The one or more mounting features 406 with the loop 422 and aperture 424 can be configured to receive suture, cable, suture anchor, fastener (e.g., bone screw) or other type of mechanical devices configured for coupling with the bone. Use of suture or cable can include cerclage technique around the humeral shaft and/or around the orthopedic implant, for example. The arms 420 can be angulated/oriented to interface with a curvature of the bone. The arms 420 can additionally be configured to flex against and conform with the outer surface of the bone, for example. Thus, the arms 420 can be formed of a shape memory or other flexible/conforming material if desired.

FIG. 4B shows an example arrangement for the onboard electronics 404 carried by the housing 402. The onboard electronics 404 may be used to generate, store, or send data. The processing circuitry 412 can be electrically coupled so as to be in electronic communication with the communication circuitry 408, the one or more sensors 410, the battery 414 and the memory 415. These components can be electrically coupled together as desired.

The communication circuitry 408 can comprise an antenna 426, for example, configured for wireless communication with the controller 208 (FIG. 2) or other electronic device external to the patient. The present invention contemplates the antenna 426 can be configured for communication on various frequency bands such as those for Bluetooth® technology (e.g., between 2400 to 2483.5 MHz), MICS (Medical Implant Communication Service) frequency band (e.g., between 402 and 405 MHz), Wi-Fi, Wi-Fi direct, via an RFID or other NFC technology, or other known dedicated modality using frequency bands approved for wireless electronic communication. The communication circuitry 408 can be electrically coupled to receive and transmit data (signals) from the processing circuitry 412.

The one or more sensors 410 can be arranged in either the cylindrical portion 416 or the main body portion 418. The one or more sensors 410 can be any one or combination of different types of sensors (e.g., an accelerometer, a gyroscope, a piezoelectric sensor, force sensor, thermometer, pH monitor, strain gauge, or any combination thereof, including any other sensor that can be used to detect motion within a body). The one or more sensors 410 can be configured to generate data regarding one or more characteristics (e.g., orientation, range of motion, movement, force, temperature, pH, swelling, etc.) of the patient.

The generated data can be sent directly to an external device (e.g., the controller 208 of FIG. 2) for processing and storage or can be stored in the memory 415 prior to processing by the processing circuitry 412, for example. Alternatively, or additionally, the generated data can be sent to the processing circuitry 412 for real-time processing followed by storage and/or transmission by the communication circuitry 408. The processing circuitry 412 can be configured to activate the communication circuitry 408 to send the generated data to a device (e.g., the controller 208 of FIG. 2) outside the patient.

The processing circuitry 412 can be configured in the manner discussed with regard to the controller 208 of FIG. 2 previously and may include an integrated circuit, such as a system on a chip, a field-programmable gate array (FPGA), a processor, etc.). The processing circuitry 412 can include one or more processors, microprocessors, microcontrollers, electronic control modules (ECMs), electronic control units (ECUs), programmable logic controller (PLC), or any other suitable means for electronically communicating with the sensing device 200A. The processing circuitry 412 can be configured to operate according to a predetermined algorithm or set of instructions. Such an algorithm or set of instructions can be stored in a database, can be read into an on-board memory of the processing circuitry 412, or preprogrammed onto the memory 415 or another device. The processing circuitry 412 may be operably modified to a different programmability capability (e.g., to change the data collection, output, model, etc.), for example changing what data is collected or how often the one or more sensors 410 collects data.

The communication circuitry 408, the one or more sensors 410, the processing circuitry 412, etc. can be powered by the battery 414. The battery 414 can be a primary battery or can be configured for recharging by induction wirelessly or using wired or other technique according to some example embodiments. Such induction recharging can be facilitated by the battery 414 and the sensing device 400 being mounted on the outer surface of the bone rather than being part of the orthopedic implant or being implanted within the bone, for example.

The memory 415 can be in the form of a floppy disk, hard drive, optical medium, random access memory (RAM), read-only memory (ROM), or any other suitable computer-readable storage medium commonly used in the art (each referred to as a “database”), which can be in the form of a physical, non-transitory storage medium.

FIGS. 4C and 4D show the sensing device 400 with the housing 402 intact. As shown in FIGS. 4C and 4D, the one or more mounting features 406 can be angulated relative to the main body portion 418 such as in a direction of the outer surface of the bone to facilitate interfacing and/or contact with the outer surface of the bone.

FIGS. 5A and 5B show a sensing device 500 according to another example. The sensing device 500 can include onboard electronics similar to those discussed with reference to the sensing devices 200, 200A and 400. However, the sensing device 500 can include a housing 502 and one or more mounting features 503 having a different shape. The housing 502 can have a bone facing side 504 and an outward facing side 506. The housing 502 can be shaped as a plate having a relatively small thickness T as compared with a width and length dimensions. The thickness T can be between 2 mm and 8 mm, for example. This shape can provide the sensing device 500 with a low thickness profile upon implantation. Additionally, as shown in FIGS. 5A and 5B, at least the bone facing side 504 can be curved. This curvature for the bone facing side 504 can be configured to match or otherwise generally correspond to a curvature of the outside surface of the bone, for example. According to one embodiment, the bone facing side 504 can include a porous coating 508 configured to facilitate/promote on-growth of the bone for fixation of sensing device 500 to the bone.

The shape of the bone facing side 504 can be configured to fit the natural anatomy of the patient. Thus, an asymmetric shape for the bone facing side 504 is contemplated. As an example, the bone facing side 504 can be shaped based on average anatomy derived from three- or two-dimensional scans of the relevant bone using X-Ray, MRI, CT, ultrasound or other imaging techniques. Such shaping can include use of large number of scans and the ZiBRA™ Anatomical Modeling System to analyze thousands of bones, both male and female, representing a diverse global population. Alternatively, the shape of the bone facing side 504 can be patient-specific (i.e. is constructed specifically for the patient). In either case, additive manufacturing or other fabrication techniques could be deployed for fabricating the housing 502 to allow for a satisfactory mating match of the shape of the bone facing side 504 with the outside surface of the bone.

If the housing is desired to be patient-specific, the housing 502 can be designed using computer modeling based upon the three or two-dimensional images of the relevant patient's anatomy. Such technique can make use of various CAD programs and/or software available from various vendors or developers. Scan data can be used by the manufacturer to construct an image of the patient's joint including the outer surface geometry of the bone and prepare an initial fitting of the bone facing side 504 to the bone. A surgical plan can allow for modification of the initial fitting by interactive software, which can be sent to the surgeon, or other medical practitioner, for review and modification if desired.

The one or more mounting features 503 can comprise arms 512 each forming an aperture 514 to receive a fastener, suture, cable or other mounting component. The arms 512 shape and orientation can facilitate interfacing with the outer surface of the bone. Arms 512 can be configured to abut with the outer surface of the bone, for example. However, abutting contact with the outer surface of the bone is not contemplated in all examples.

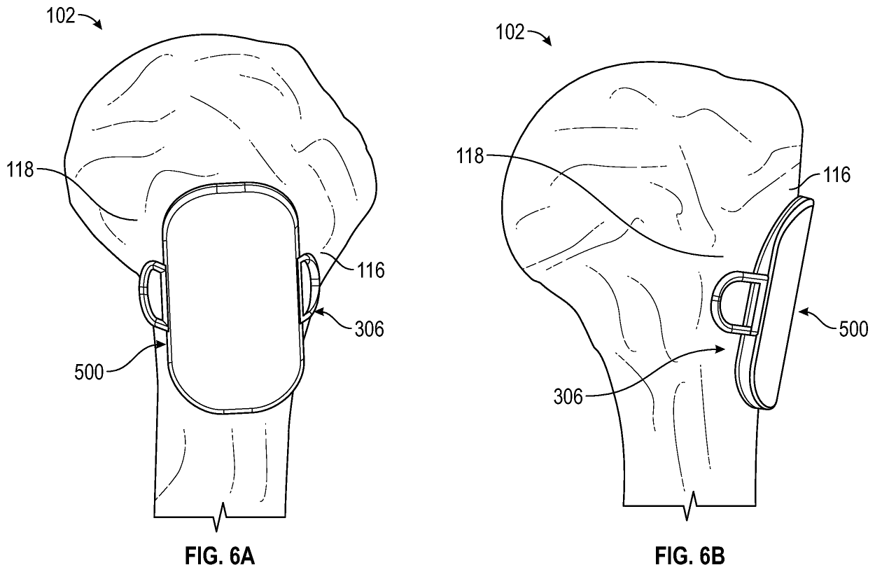

As shown in FIGS. 6A and 6B, the sensing device 500 is implanted on the outer surface 116 of the humerus 102 at the metaphysis region 118 and/or the diaphysis region thereof. Such implantation of the sensing device 500 can be on the medial side area or the lateral side area 306 of the humerus 102 as shown in FIGS. 6A and 6B.

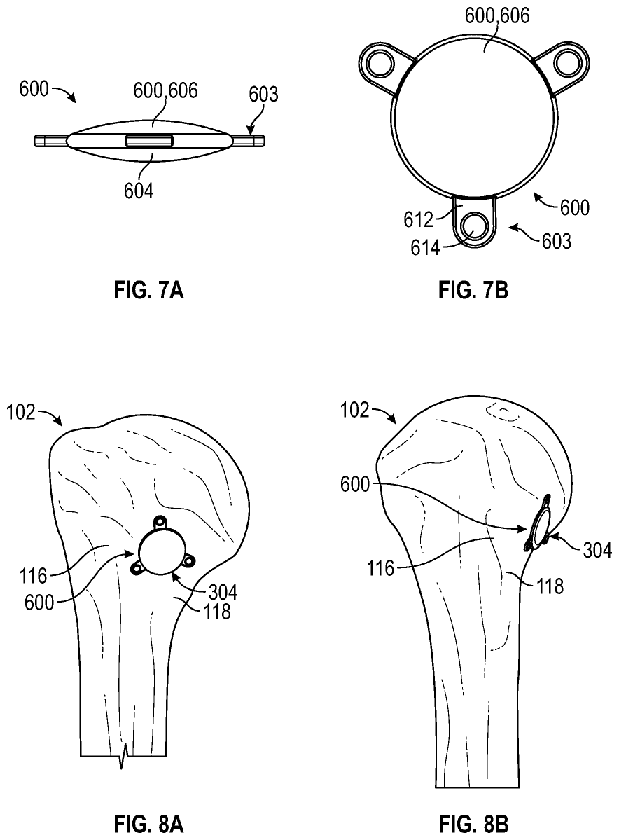

FIGS. 7A and 7B show yet another sensing device 600 that is configured for implantation on the external surface of bone such as the humerus. The sensing device 600 can include onboard electronics similar to those discussed with reference to the sensing devices 200, 200A, 400 and 500. However, the sensing device 600 can include a housing 602 and one or more mounting features 603 having a different shape. The housing 602 can have a bone facing side 604 and an outward facing side 606. The housing 602 can be shaped as a semi-sphere, sphere or disk. A thickness of the housing 602 can be between 2 mm and 8 mm, for example. This shape can provide the sensing device 600 with a relatively low thickness profile upon implantation. Additionally, as shown in FIGS. 8A and 8B, at least the bone facing side 604 can be curved. This curvature for the bone facing side 604 can be configured to match or otherwise generally correspond to a curvature of the outside surface of the bone, for example. According to one embodiment, the bone facing side 604 can include a porous coating (not shown) configured to facilitate/promote on-growth of the bone for fixation of sensing device 600 to the bone.

The shape of the bone facing side 604 can be configured to fit the natural anatomy of the patient and can be designed using the techniques discussed above such as with the ZiBRA™ Anatomical Modeling System or can be patient-specific (i.e. is constructed specifically for the patient).

The one or more mounting features 603 can comprise arms 612 each forming an aperture 614 to receive a fastener, suture, cable or other mounting component. The arms 612 shape and orientation can facilitate interfacing with the outer surface of the bone. Arms 612 can be configured to abut with the outer surface of the bone, for example. However, abutting contact with the outer surface of the bone is not contemplated in all examples.

FIGS. 8A and 8B show the sensing device 600 is implanted on the outer surface 116 of the humerus 102 at the metaphysis region 118 and/or the diaphysis region thereof. Such implantation of the sensing device 600 can be on the medial side area or the medial to lesser tuberosity 304 of the humerus 102 as shown in FIGS. 8A and 8B.

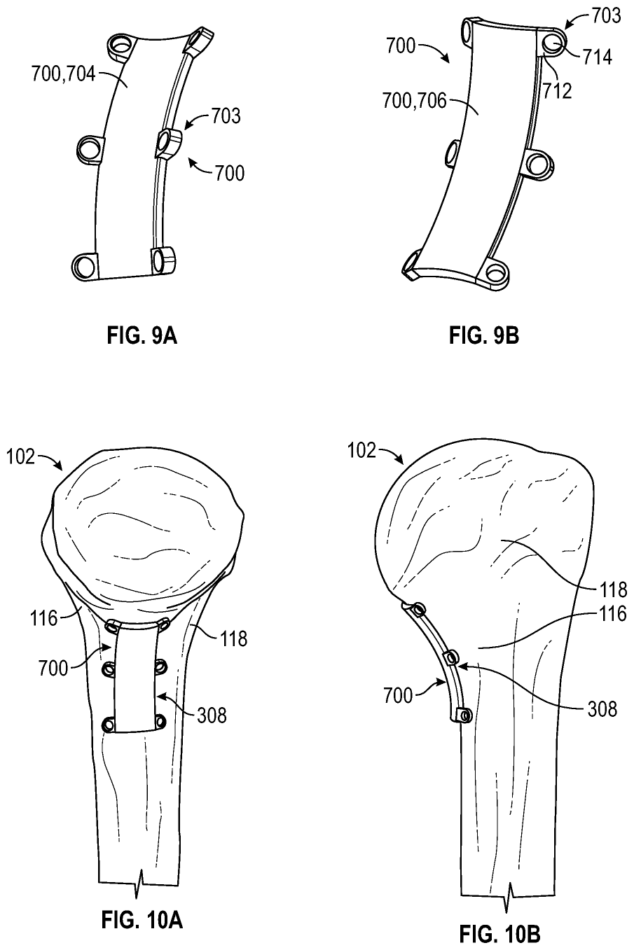

FIGS. 9A and 9B show yet another sensing device 700 that is configured for implantation on the external surface of bone such as the humerus. The sensing device 700 can include onboard electronics similar to those discussed with reference to the sensing devices 200, 200A, 400, 500 and 600. However, the sensing device 700 can include a housing 702 and one or more mounting features 703 having a different shape. The housing 702 can have a bone facing side 704 and an outward facing side 706. The housing 702 can be shaped curved in two or more directions such as proximal-distal and medial lateral. A thickness of the housing 702 as measured between the bone facing side 704 and the outward facing side 706 can be between 2 mm and 8 mm, for example. This shape can provide the sensing device 700 with a relatively low thickness profile upon implantation. The curvature for the bone facing side 704 can be configured to match or otherwise generally correspond to a curvature of the outside surface of the bone, for example. According to one embodiment, the bone facing side 704 can include a porous coating (not shown) configured to facilitate/promote on-growth of the bone for fixation of sensing device 700 to the bone.

The shape of the bone facing side 704 can be configured to fit the natural anatomy of the patient and can be designed using the techniques discussed above such as with the ZiBRA™ Anatomical Modeling System or can be patient-specific (i.e. is constructed specifically for the patient).

The one or more mounting features 703 can comprise arms 712 each forming an aperture 714 to receive a fastener, suture, cable or other mounting component. The arms 712 shape and orientation can facilitate interfacing with the outer surface of the bone. Arms 712 can be configured to abut with the outer surface of the bone, for example. However, abutting contact with the outer surface of the bone is not contemplated in all examples.

FIGS. 10A and 10B show the sensing device 700 is implanted on the outer surface 116 of the humerus 102 at the metaphysis region 118 and/or the diaphysis region thereof. Such implantation of the sensing device 700 can be on the medial side area 308 of the humerus 102 as shown in FIGS. 10A and 10B.

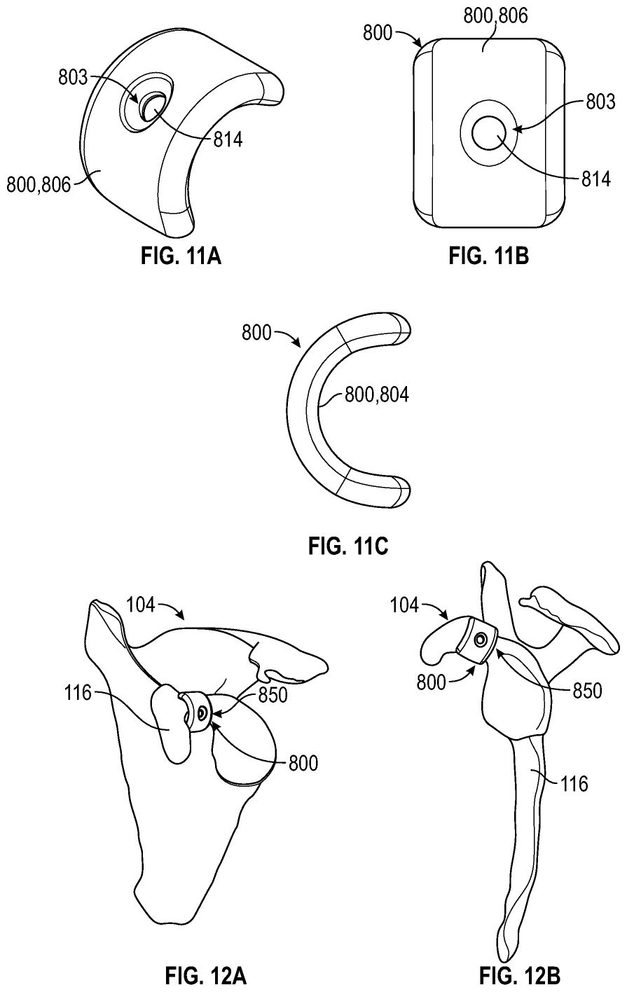

FIGS. 11A-11C show yet another sensing device 800 that is configured for implantation on the external surface of bone such as the scapula. The sensing device 800 can include onboard electronics similar to those discussed with reference to the sensing devices 200, 200A, 400, 500, 600 and 700. However, the sensing device 800 can include a housing 802 and one or more mounting features 803 having a different shape. The housing 802 can have a bone facing side 804 and an outward facing side 806. The housing 802 can be curved or actuate in a U-shape have a dramatic curvature. A thickness of the housing 802 as measured between the bone facing side 804 and the outward facing side 806 can be between 2 mm and 8 mm, for example. This shape can provide the sensing device 800 with a relatively low thickness profile upon implantation. The curvature for the bone facing side 804 can be configured to match or otherwise generally correspond to a curvature of the outside surface of the bone, for example. According to one embodiment, the bone facing side 804 can include a porous coating (not shown) configured to facilitate/promote on-growth of the bone for fixation of sensing device 800 to the bone.

The shape of the bone facing side 804 can be configured to fit the natural anatomy of the patient and can be designed using the techniques discussed above such as with the ZiBRA™ Anatomical Modeling System or can be patient-specific (i.e. is constructed specifically for the patient).

The one or more mounting features 803 can comprise an aperture 814 in the housing 802 to receive a fastener, suture, cable or other mounting component. FIGS. 12A and 12B show the sensing device 800 is implanted on the outer surface 116 of the scapula 104 at a base of the coracoid 850.

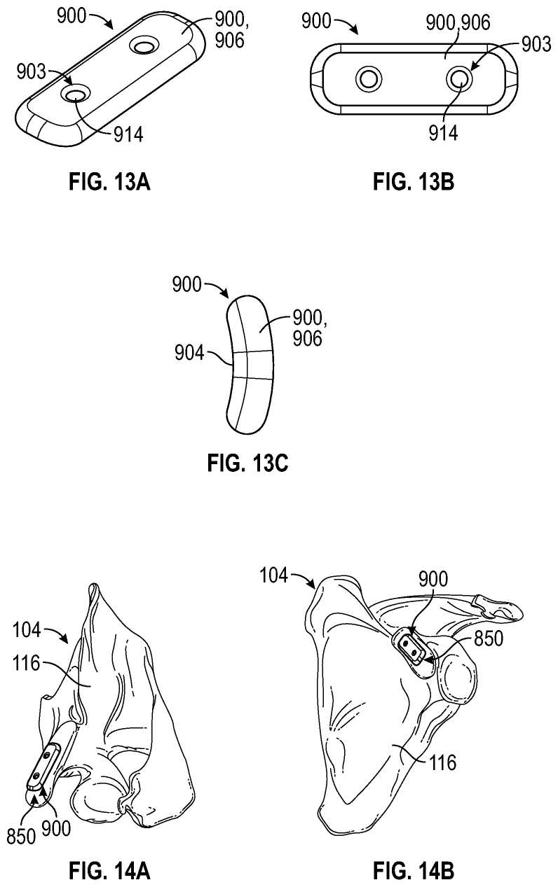

FIGS. 13A-13C show yet another sensing device 900 that is configured for implantation on the external surface of bone such as the scapula. The sensing device 900 can include onboard electronics similar to those discussed with reference to the sensing devices 200, 200A, 400, 500, 600, 700 and 800. However, the sensing device 900 can include a housing 902 and one or more mounting features 903 having a different shape. The housing 902 can have a bone facing side 904 and an outward facing side 906. The housing 902 can be slightly curved along the bone facing side 904 and can have an elongate length. A thickness of the housing 902 as measured between the bone facing side 904 and the outward facing side 906 can be between 2 mm and 8 mm, for example. This shape can provide the sensing device 900 with a relatively low thickness profile upon implantation. The curvature for the bone facing side 904 can be configured to match or otherwise generally correspond to a curvature of the outside surface of the bone, for example. According to one embodiment, the bone facing side 904 can include a porous coating (not shown) configured to facilitate/promote on-growth of the bone for fixation of sensing device 900 to the bone.

The shape of the bone facing side 904 can be configured to fit the natural anatomy of the patient and can be designed using the techniques discussed above such as with the ZiBRA™ Anatomical Modeling System or can be patient-specific (i.e. is constructed specifically for the patient).

The one or more mounting features 903 can comprise an aperture 914 in the housing 802 to receive a fastener, suture, cable or other mounting component. FIGS. 14A and 14B show the sensing device 900 is implanted on the outer surface 116 of the scapula 104 at a proximal side of the coracoid 850.

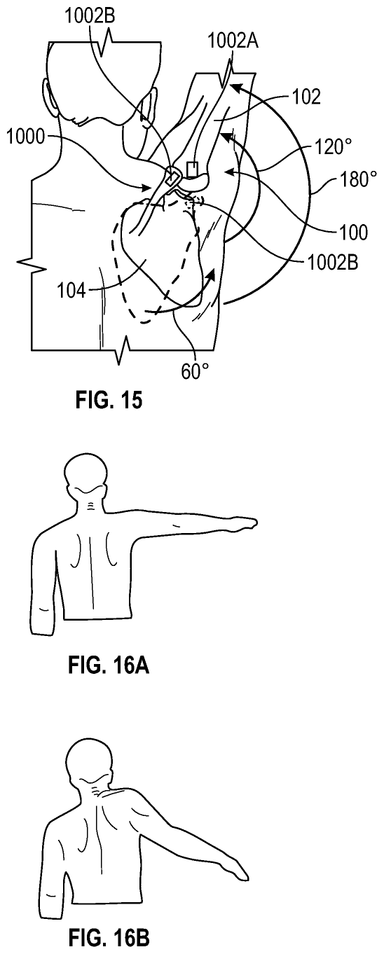

FIG. 15 shows a system 1000 including at least two sensors 1002A and 1002B. At least one of the two sensors 1002A or 1002B can be an externally bone mounted sensor as previously discussed herein. FIG. 15 shows the sensor 1002A mounted to the humerus 102 and the sensor 1002B mounted to the scapula 104. The sensor 1002B can be configured as the sensors 900 or 800 discussed herein, for example. Similarly, the sensor 1002A can be any of the sensors shown in FIGS. 1-10B for mounting to the external bone surface of the humerus 102.

As shown in FIG. 15, the sensors 1002A and 1002B can work in tandem to provide data about the function of the shoulder joint 100. FIG. 15 depicts motion of the shoulder joint 100 and corresponding movement of sensor 1002B (from location in phantom to the present raised arm location).

One or both of the sensors 1002A and/or 1002B can be configured as an inertial measurement unit (IMU). One challenge is that an IMU only measures angular changes relative to gravity. Clinically, these measurements are irrelevant without the assumption that the subject's torso is in alignment with the gravity vector know by the IMU. By mounting an IMU (the sensors 1002A and 1002B) to the scapula and the humerus, more exact glenohumeral joint motion can be calculated based on the common gravity reference. Further, if an assumption is made about the patient's torso in alignment with the gravity vector, scapular motion can be calculated/measured which contributes to the full range of motion experienced by the patient. It should be noted that the sensors 1002A and 1002B can both be powered by a single battery that is contained in either sensor 1002A or 1002B. For example, the sensor 1002A could be directly connected to sensor 1002B via a lead or could charge the sensor 1002B via induction. This could reduce the size of the sensor 1002A or 1002B as a battery need not be included with the IMU.

Thus, glenohumeral joint motion calculated by taking simultaneous measurements with both IMUs (both sensors 1002A and 1002B). From the data gathered by the sensors 1002A and 1002B, an external electronic device such as a controller can calculate scapular motion utilized to achieve full abduction as shown in FIG. 15. As an example, the sensor 1002A on humerus 102 measures 180° of motion relative to gravity (abduction). The sensor 1002B on the scapula 104 measures 60° of motion relative to gravity (scapular motion's contribution to total abduction). The difference (180 degrees−60 degrees) of 120 degrees is the glenohumeral joint motion. This methodology assumes if IMUs are being used that the patient's torso is upright (in alignment with gravity) to obtain proper measurement.

In a clinical application, excessive scapular motion during abduction can indicate that a patient has a deteriorating/torn rotator cuff. Detecting excessive scapular motion could be used to indicate when a patient with an anatomic total shoulder should be converted to a reverse. Scapular motion patterns could also be used to understand shoulder tension (too loose/tight) and implant loosening.

FIGS. 16A and 16B illustrate a potential clinical application that could be measured using the sensors 1002A and 1002B of FIG. 15 working in tandem. FIG. 16A depicts abduction with a healthy rotator cuff. FIG. 16B shows abduction with a torn rotator cuff. In FIG. 16A, 90 degree abduction is achieved, with this movement a humeral sensor would be positioned at 90 degrees and the scapular sensor would be at 20 degrees. Glenohumeral joint motion would be 70 degrees (90 degrees−20 degrees). 70/90=78% of motion from glenohumeral joint.

Turning to FIG. 16B, with the joint torn 70 degrees of abduction is achieved. The humeral sensor would measure 70 degrees. The scapular sensor would measure 30 degrees (see movement of scapula of patient as illustrated in FIG. 16B). The glenohumeral joint motion would be 40 degrees (70 degrees−30 degrees). 40/70=57% of motion from glenohumeral joint.

A reduction in % of total motion contribution from the glenohumeral joint motion indicates a rotator cuff issue as portrayed in FIGS. 16A and 16B.

### Notes and Examples

The following, non-limiting examples (sometimes referred to as techniques below), detail certain aspects of the present subject matter to solve the challenges and provide the benefits discussed herein, among others.

Example 1 is a sensing device configured for implantation on an outer surface of a bone of a patient, the sensing device optionally including: a housing; one or more features configured to couple the housing against the outer surface of the bone of the patient; and onboard electronics carried by the housing, the onboard electronics including: communication circuitry; one or more sensors configured to generate data regarding one or more characteristics of the patient; and processing circuitry configured to activate the communication circuitry to send the generated data to a device outside the patient.

Example 2 is the sensing device of Example 1, wherein the onboard electronics further optionally include a battery configured to provide power to one or more of the one or more sensors and the processing circuitry, wherein the battery is configured for recharging by induction.

Example 3 is the sensing device of any one or combination of Examples 1-2, wherein the communication circuitry optionally is part of a transdermal lead.

Example 4 is the sensing device of any one or combination of Examples 1-3, wherein the communication circuitry includes optionally an antenna configured for communication over at least one of a frequency band approved for medical communication or another frequency band approved for wireless communication.

Example 5 is the sensing device of any one or combination of Examples 1-4, wherein the one or more sensors optionally include one or more of a gyroscope, an accelerometer, a thermometer, a pH monitor or a force sensor.

Example 6 is the sensing device of any one or combination of Examples 1-5, wherein optionally at least the housing and the one or more features are configured for implantation at one or more of: a biceps groove of a humerus; a medial to lesser tuberosity of the humerus; a lateral side of the humerus extending from adjacent a humeral head to a humeral neck; or a medial side of the humerus below an articular margin.

Example 7 is the sensing device of any one or combination of Examples 1-6, wherein optionally the one or more features include arms each of the arms with an aperture configured to receive one or more of a suture, cable, bone screw or porous coating.

In Example 8 is the sensing device of any one or combination of Examples 1-7, wherein the outer surface is part of a metaphysis region of the bone.

Example 9 is a system configured to be implanted into a patient, the system optionally including: an orthopedic implant configured for coupling to a resected surface of a bone of the patient, wherein the orthopedic implant is configured to replicate a portion of the bone that forms a joint of the patient; and a sensing device external of the orthopedic implant, wherein the sensing device is configured for implantation on an outer surface of the bone of the patient and including one or more sensors configured to generate data regarding one or more characteristics of the patient.

Example 10 is the system of Example 9, wherein optionally the sensing device is configured for mounting to one or more of: a biceps groove of a humerus; a medial to lesser tuberosity of the humerus; a lateral side of the humerus extending from adjacent a humeral head to a humeral neck; or a medial side of the humerus below an articular margin.

Example 11 is the system of any one or combination of Examples 9-10, wherein optionally the one or more sensors are configured to measure one or more of: acceleration, angular velocity, orientation, pH, force or temperature adjacent the orthopedic implant when implanted on the outer surface of a metaphysis region of the bone.

Example 12 is the system of any one or combination of Examples 9-11, wherein the sensing device optionally includes: communication circuitry; and processing circuitry configured to activate the communication circuitry to send generated data to a device outside the patient.

Example 13 is the system of any one or combination of Examples 9-12, wherein the communication circuitry optionally includes one of: a transdermal lead; or an antenna configured for wireless communication.

Example 14 is the system of any one or combination of Examples 9-13, wherein the sensing device optionally includes a battery configured to provide power to the one or more sensors, wherein the battery is configured for recharging by induction.

Example 15 is the system of any one or combination of Examples 9-14, wherein optionally the sensing device is configured for implantation on the outer surface of a metaphysis region of the bone in close proximity to the orthopedic implant and the joint.

Example 16 is the system of any one or combination of Examples 9-15, further optionally including one or more of: a suture configured to be received by the sensing device and configured to wrap around at least a portion of the bone; a suture anchor configured to anchor into the bone; a cable configured to be received by the sensing device and configured to wrap around at least a portion of the bone; a bone screw configured to be received by the sensing device and configured for fixation in the bone; or a porous coating on the sensing device, wherein the porous coating configured to promote on-growth of the bone for fixation of the sensing device with the bone.

Example 17 is the system of any one or combination of Examples 9-16, wherein the sensing device optionally has a patient-specific configuration to mate with and mount to the external surface at one of a biceps groove of a humerus, a medial to lesser tuberosity of the humerus, a lateral side of the humerus extending from adjacent a humeral head to ae humeral neck or a medial side of the humerus below an articular margin.

Example 18 is a method of monitoring one or more characteristics of a patient, the method optionally including: implanting an orthopedic implant to replicate a portion of a joint of a patient; implanting a sensing device spaced from the orthopedic implant on an outer surface of a metaphysis region of a bone adjacent the joint; operating the sensing device while implanted on the outer surface of the metaphysis region to generate data regarding the one or more characteristics of the patient; and communicating the generated data from the sensing device to a remote computing device external of the patient.

Example 19 is the method of Example 18, wherein optionally implanting the sensing device external of the orthopedic implant on the outer surface of the metaphysis region of the bone adjacent the joint includes implanting the sensing device at one of: a biceps groove of a humerus; a medial to lesser tuberosity of the humerus; a lateral side of the humerus extending from adjacent a humeral head to a humeral neck; or a medial side of the humerus below an articular margin.

Example 20 is the method of any one or combination of Examples 18-19, wherein optionally implanting the sensing device external of the orthopedic implant on the outer surface of the metaphysis region of the bone adjacent the joint includes at least one of: suturing using a cerclage technique around a shaft of a humerus and/or the orthopedic implant, cabling using the cerclage technique around the shaft of the humerus and/or the orthopedic implant, fixating the sensing device to the outer surface with one or more bone screws or coupling with a porous coating.

Example 21 is the method of any one or combination of Examples 18-20, further optionally including recharging a battery of the sensing device using one of a transdermal lead or induction.

Example 22 is the method of any one or combination of Examples 18-21, wherein optionally communicating the generated data from the sensing device is via the transdermal lead.

Example 23 is the method of any one or combination of Examples 18-22, wherein optionally communicating the generated data from the sensing device to a remote computing device external of the patient includes wireless communication.

In Example 24, the apparatuses system, or method of any one or any combination of Examples 1-23 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

The terms “adjacent” and “close proximity” as used herein mean spaced from another feature or component. The distance of such spacing can vary according to the size of the joint, the relative positioning of components/feature and other factors.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A sensing device configured for implantation on an outer surface of a bone of a patient, the sensing device comprising:
a housing;
one or more features configured to couple the housing against the outer surface of the bone of the patient; and
onboard electronics carried by the housing, the onboard electronics comprising:
communication circuitry;
one or more sensors configured to generate data regarding one or more characteristics of the patient; and
processing circuitry configured to activate the communication circuitry to send the generated data to a device outside the patient.

2. The sensing device of claim 1, wherein the onboard electronics further include a battery configured to provide power to one or more of the one or more sensors and the processing circuitry, wherein the battery is configured for recharging by induction.

3. The sensing device of claim 1, wherein the communication circuitry is part of a transdermal lead.

4. The sensing device of claim 1, wherein the communication circuitry includes an antenna configured for communication over at least one of a frequency band approved for medical communication or another frequency band approved for wireless communication.

5. The sensing device of claim 1, wherein the one or more sensors comprise one or more of a gyroscope, an accelerometer, a thermometer, a pH monitor or a force sensor.

6. The sensing device of claim 1, wherein at least the housing and the one or more features are configured for implantation at one or more of:
a biceps groove of a humerus;
a medial to lesser tuberosity of the humerus;
a lateral side of the humerus extending from adjacent a humeral head to a humeral neck; or
a medial side of the humerus below an articular margin.

7. The sensing device of claim 1, wherein the one or more features comprise arms each of the arms with an aperture configured to receive one or more of a suture, cable, bone screw or porous coating.

8. The sensing device of claim 1, wherein the outer surface is part of a metaphysis region of the bone.

9. A system configured to be implanted into a patient, the system comprising:
an orthopedic implant configured for coupling to a resected surface of a bone of the patient, wherein the orthopedic implant is configured to replicate a portion of the bone that forms a joint of the patient; and
a sensing device external of the orthopedic implant, wherein the sensing device is configured for implantation on an outer surface of the bone of the patient and including one or more sensors configured to generate data regarding one or more characteristics of the patient.

10. The system of claim 9, wherein the sensing device is configured for mounting to one or more of:
a biceps groove of a humerus;
a medial to lesser tuberosity of the humerus;
a lateral side of the humerus extending from adjacent a humeral head to a humeral neck; or
a medial side of the humerus below an articular margin.

11. The system of claim 9, wherein the one or more sensors are configured to measure one or more of: acceleration, angular velocity, orientation, pH, force or temperature adjacent the orthopedic implant when implanted on the outer surface of a metaphysis region of the bone.

12. The system of claim 9, wherein the sensing device includes:
communication circuitry; and
processing circuitry configured to activate the communication circuitry to send generated data to a device outside the patient.

13. The system of claim 12, wherein the communication circuitry comprises one of:
a transdermal lead; or
an antenna configured for wireless communication.

14. The system of claim 9, wherein the sensing device includes a battery configured to provide power to the one or more sensors, wherein the battery is configured for recharging by induction.

15. The system of claim 9, wherein the sensing device is configured for implantation on the outer surface of a metaphysis region of the bone in close proximity to the orthopedic implant and the joint.

16. The system of claim 9, further comprising one or more of:
a suture configured to be received by the sensing device and configured to wrap around at least a portion of the bone;
a suture anchor configured to anchor into the bone;
a cable configured to be received by the sensing device and configured to wrap around at least a portion of the bone;
a bone screw configured to be received by the sensing device and configured for fixation in the bone; or
a porous coating on the sensing device, wherein the porous coating configured to promote on-growth of the bone for fixation of the sensing device with the bone.

17. The system of claim 9, wherein the sensing device has a patient-specific configuration to mate with and mount to the external surface at one of a biceps groove of a humerus, a medial to lesser tuberosity of the humerus, a lateral side of the humerus extending from adjacent a humeral head to ae humeral neck or a medial side of the humerus below an articular margin.

18. A method of monitoring one or more characteristics of a patient, the method comprising:
implanting an orthopedic implant to replicate a portion of a joint of a patient;
implanting a sensing device spaced from the orthopedic implant on an outer surface of a metaphysis region of a bone adjacent the joint;
operating the sensing device while implanted on the outer surface of the metaphysis region to generate data regarding the one or more characteristics of the patient; and
communicating the generated data from the sensing device to a remote computing device external of the patient.

19. The method of claim 18, wherein implanting the sensing device external of the orthopedic implant on the outer surface of the metaphysis region of the bone adjacent the joint includes implanting the sensing device at one of:
a biceps groove of a humerus;
a medial to lesser tuberosity of the humerus;
a lateral side of the humerus extending from adjacent a humeral head to a humeral neck; or
a medial side of the humerus below an articular margin.

20. The method of claim 18, wherein implanting the sensing device external of the orthopedic implant on the outer surface of the metaphysis region of the bone adjacent the joint includes at least one of: suturing using a cerclage technique around a shaft of a humerus and/or the orthopedic implant, cabling using the cerclage technique around the shaft of the humerus and/or the orthopedic implant, fixating the sensing device to the outer surface with one or more bone screws or coupling with a porous coating.
